(19) United States
(12) Patent Application Publication
(10) Pub. No.: US 2011/0048237 A1
(43) Pub. Date: Mar. 3, 2011

(54) Air cleaning humidifier

(75) Inventors: Jong Chul Ahn, Yongin-si (KR); Jung Ho Kim, Suwon-si (KR); Jun Eui Chang, Suwon-City (KR)

(73) Assignee: SAMSUNG ELECTRONICS CO., LTD., Suwon-si (KR)

(21) Appl. No.: 12/805,195

(22) Filed: Jul. 16, 2010

(30) Foreign Application Priority Data
Aug. 28, 2009 (KR) 10-2009-80319

(51) Int. Cl. F24F 6/02; A61L 9/00; B01F 3/04; F24F 6/06; F24F 6/08

(52) U.S. Cl. 96/223; 261/80; 261/141

(57) ABSTRACT

An air cleaning humidifier which minimizes interference or resistance between cleaned air and a disc assembly mounted to the humidifier to perform the humidifying function while the cleaned air is being passed through the disc assembly by a ventilation fan. The air cleaning humidifier includes a body frame having an air discharge port, a water tub disposed in the body frame, a ventilation fan generating an air current in the body frame, and a disc assembly arranged around the ventilation fan and rotated with a part thereof submerged in the water tub. The disc assembly includes a ring portion forming a circumferential body thereof and a cut portion formed by cutting a part of the ring portion and disposed near the air discharge port.

### CROSS-REFERENCE TO RELATED APPLICATIONS

This application claims the benefit of Korean Patent Application No. 2009-0080319, filed on Aug. 28, 2009 in the Korean Intellectual Property Office, the disclosure of which is incorporated herein by reference.

### BACKGROUND

1. Field

Embodiments relate to an air cleaning humidifier equipped with a disc assembly and capable of cleaning and humidifying the interior air.

2. Description of the Related Art

Generally, a humidifier is used to maintain a proper level of humidity in an interior space and thereby prevent a variety of respiratory ailments.

There are various types of humidifiers employing different methods. In particular, a vaporizing-type humidifier, has been recently developed, has a disc assembly and operates in a manner that air cleaned by an air cleaning filter is passed through the disc assembly soaked with water and discharged to the outside through a discharge port, during which natural humidification is performed.

The vaporizing-type humidifier selectively carries out an air cleaning and humidifying mode performing both air cleaning and humidifying simultaneously, and an air cleaning mode performing air cleaning only.

During the air cleaning mode, while the cleaned air is being passed through the disc assembly provided for the air humidifying function by a ventilation fan mounted at the humidifier, the wind velocity may be reduced due to resistance of the disc assembly.

### SUMMARY

Therefore, it is an aspect to provide an air cleaning humidifier which minimizes interference or resistance between cleaned air and a disc assembly mounted to the humidifier to perform the humidifying function while the cleaned air is being passed through the disc assembly by a ventilation fan.

Additional aspects will be set forth in part in the description which follows and, in part, will be apparent from the description, or may be learned by practice of the invention.

The foregoing and/or other aspects are achieved by providing an air cleaning humidifier including a body frame having an air discharge port; a water tub disposed in the body frame; a ventilation fan generating an air current in the body frame; and a disc assembly arranged around the ventilation fan and rotated with a part thereof submerged in the water tub, wherein the disc assembly includes a ring portion forming a circumferential body thereof and a cut portion formed by cutting out a part of the ring portion and disposed adjacent the air discharge port.

The cut portion may be formed by cutting out a part of the ring portion in a fan-shape.

The air cleaning humidifier may further include a driving unit driving the disc assembly to transmit a rotational force to the disc assembly.

The disc assembly may include a side plate equipped with a gear unit, and the driving unit may be engaged with the gear unit.

The driving unit may rotate the disc assembly forward and backward in a manner that only the ring portion of the disc assembly is submerged in the water tub while the cut portion is not submerged.

The air cleaning humidifier may further include a supporting member formed in the water tub to support the disc assembly.

A center of rotation of the disc assembly may be axially aligned with a center of rotation of the ventilation fan, and the disc assembly may be received in the body frame so that thickness portions of the disc assembly and the ventilation fan are overlapped with each other.

The disc assembly may include an opening having a greater diameter than an outer diameter of the ventilation fan so that the disc assembly fan may be inserted in the disc assembly.

The disc assembly may include at least two side plates, a plurality of discs disposed between the side plates, and spacers to maintain predetermined intervals among the discs.

The body frame may include an air suction port formed at a rear surface thereof to draw air.

The cut portion may be disposed nearest to the air discharge port when an air cleaning mode is selected.

The body frame may further include a heater to heat the air humidified by the disc assembly.

The body frame may further include a sterilizer to sterilize the air humidified by the disc assembly.

The foregoing and/or other aspects are achieved by providing an air cleaning humidifier including a body frame having an air suction port and an air discharge port; a water tub connected to a lower part of the body frame; a disc assembly rotated while partially submerged in the water tub; and a ventilation fan rotated in the state of being aligned with a center of rotation of the disc assembly, to draw in air through the air suction port and discharge the air through the air discharge port, wherein the disc assembly includes a cut portion formed by cutting out a part of a circumference of the disc assembly so that an air current generated by the ventilation fan is smoothly moved to the air discharge port.

The air cleaning humidifier may further include a driving unit that rotates the disc assembly forward and backward in a manner that the cut portion of the disc assembly is not submerged in the water tub.

The driving unit may include a step motor which rotates the disc assembly forward and then backward by a predetermined input angle.

As described above, the air cleaning humidifier according to the exemplary embodiments may be able to minimize interference between an air current generated by a ventilation fan and a disc assembly by having a cut portion at the disc assembly. Accordingly, the ventilation efficiency may be enhanced.

### BRIEF DESCRIPTION OF THE DRAWINGS

These and/or other aspects will become apparent and more readily appreciated from the following description of the embodiments, taken in conjunction with the accompanying drawings of which:

FIG. 1 is a perspective view showing the exterior appearance of an air cleaning humidifier according to an embodiment;

FIG. 2 is an exploded perspective view of the air cleaning humidifier shown in FIG. 1;

FIG. 3 is a perspective view showing an assembled state of a disc assembly of the air cleaning humidifier;

FIG. 4 is a side-sectional view of the disc assembly of FIG. 3;

FIG. 5 is a front view of the disc assembly of FIG. 3;

FIG. 6 is a partially enlarged view of the disc assembly of FIG. 3;

FIG. 7A through FIG. 7E are sectional views showing the rotational operation of the disc assembly.

### DETAILED DESCRIPTION

Reference will now be made in detail to the exemplary embodiments, examples of which are illustrated in the accompanying drawings, wherein like reference numerals refer to like elements throughout.

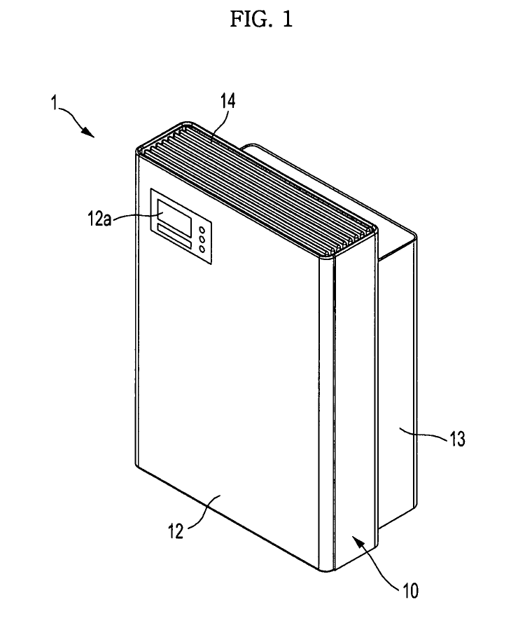

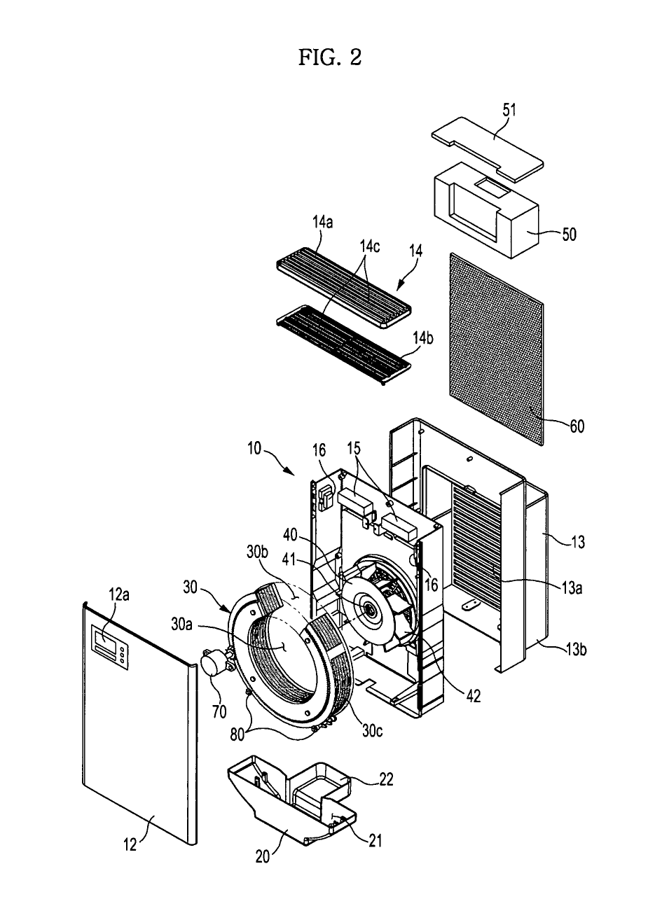

FIG. 1 is a perspective view showing the exterior appearance of an air cleaning humidifier according to an embodiment. FIG. 2 is an exploded perspective view of the air cleaning humidifier shown in FIG. 1.

As shown in FIG. 1 and FIG. 2, the air cleaning humidifier 1 includes a body frame 10 defining the exterior appearance thereof, a water tub 20 disposed in the body frame 10 to store water, a disc assembly 30 rotated while partially submerged in the water tub 20, and a ventilation device 40 blowing air to the disc assembly 30.

The body frame 10 includes a main body 11 that is a framework of the air cleaning humidifier 1, a front cover 12 forming the front exterior appearance of the air cleaning humidifier 1, a rear cover 13 forming the rear exterior appearance of the air cleaning humidifier 1, and a discharge grill 14 disposed at an upper side of the body frame 10 and formed with an air discharge port 14c.

The main body 11 is equipped with various components including the water tub 20, the disc assembly 30 and the ventilation device 40, serving as a framework that supports the entire air cleaning humidifier 1.

The water tub 20 has a space to hold water. A lower part of the disc assembly 30 is disposed in a water holding part 21 of the water tub 20 to be submerged in water. That is, the disc assembly 30 is soaked in the water stored in the water holding part 21 of the water tub 20. In addition, a water supply part 22 is provided to the water tub 20 to be supplied with water from a water tank that will be described later.

The disc assembly 30 has a substantially ring shape.

The ventilation device 40 includes a ventilation motor 41 supplying a driving force, and a ventilation fan 42 receiving the driving force from the ventilation motor 41 and thereby generating an air current. The ventilation fan 42 generates the air current inside the air cleaning humidifier 1 so that air is drawn in through an air suction port 13a and then discharged through the air discharge port 14c.

A heater 15 is mounted at an upper part of the main body 11. The heater 15 heats humidified air being discharged to the outside, when necessary, thereby producing warm humidified air.

A sterilizer 16 is mounted at both sides of an upper part of the main body 11 to exterminate harmful germs from the humidified air, such that clean humidified air is discharged to the outside of the air cleaning humidifier 1.

The front cover 12 has a substantially rectangular shape and is connected to a front side of the main body 11. A surface of the front cover 12 may be decorated with various pictures or figures. An operation unit 12a is formed at an upper part of the front cover 12 in order for operation of the air cleaning humidifier 1.

The operation unit 12a has various buttons to allow a user to operate the air cleaning humidifier 1. The operation unit 12a may be a button-operated type or a touch-operated type.

The rear cover 13 is connected with a rear side of the frame body 11, including a water tank receiving part 13b to receive a water tank 50. Also, a plurality of the air suction ports 13a are formed at the rear cover 13, such that dry air of the interior space is drawn in through the air inlets 13a.

The water tank receiving part 13b is protruded from a rear surface of the rear cover 13 and opened at an upper side. An inner cover 13c is mounted in the water tank receiving part 13b and then the water tank 50 is received on the inner cover 13c.

The water tank 50 stores water to be used for humidification and supplies a proper quantity of the water to the water holding part 21 of the water tub 20 through the water supply part 22. The water tank 50 is mounted in the form of a cassette in the air cleaning humidifier 1.

After the water tank 50 is mounted in the air cleaning humidifier 1, an upper part of the water tank 50 is covered with a water tank cover 51. A filter 60 is mounted at an inner rear surface of the water tank receiving part 13b so as to remove impurities from the air drawn in from the outside.

The discharge grill 14 is connected to the upper side of the body frame 10 and is divided into an external discharge grill 14a mounted at the outer part of the body frame 10 and an internal discharge grill 14b mounted at the inner part of the external discharge grill 14a.

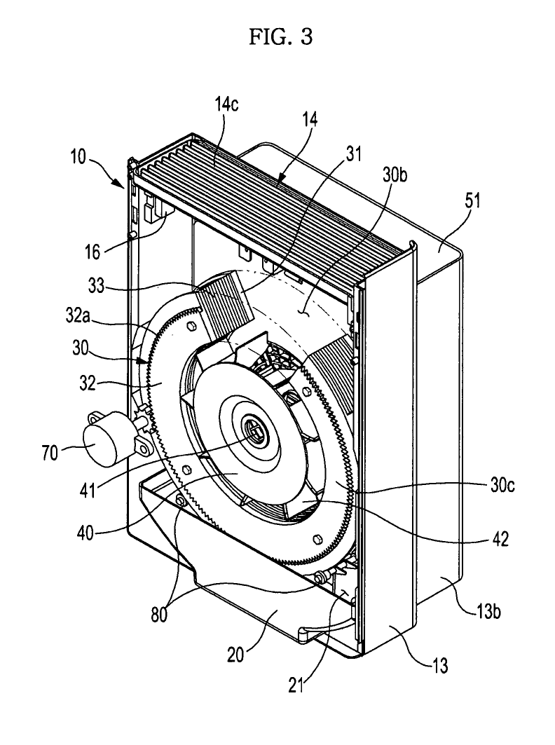

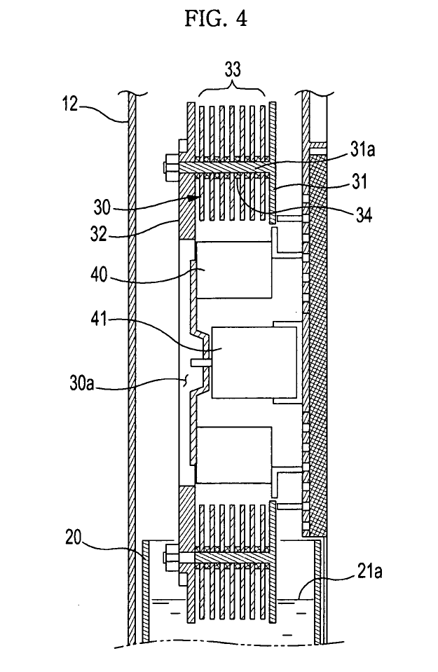

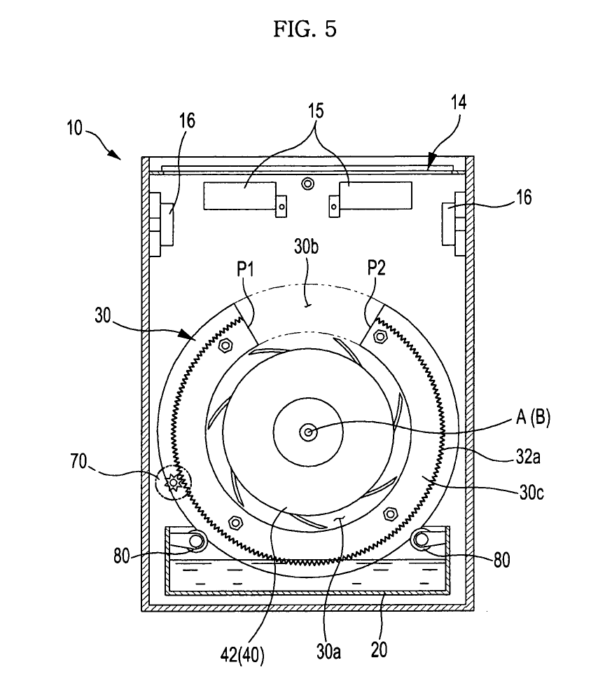

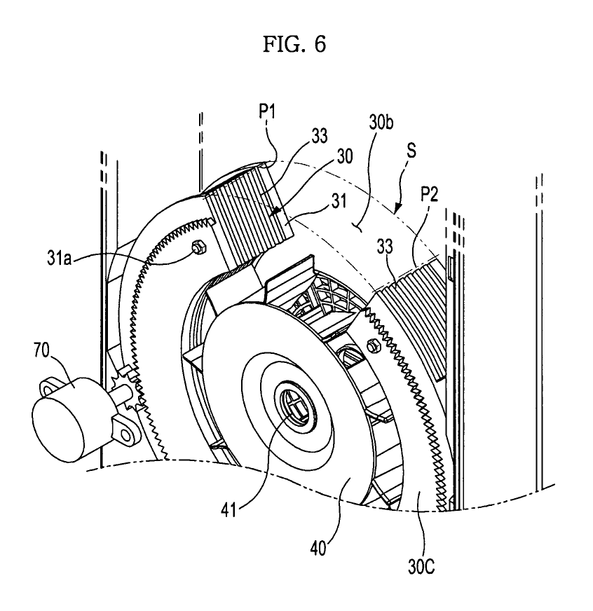

Hereinafter, the disc assembly according to the exemplary embodiment will be described with reference to FIGS. 3 to 6. FIG. 3 is a perspective view showing an assembled state of the disc assembly. FIG. 4 is a side-sectional view of the disc assembly of FIG. 3. FIG. 5 is a front view of the disc assembly of FIG. 3. FIG. 6 is a partially enlarged view of the disc assembly of FIG. 3.

Referring to FIGS. 3 to 6, in the air cleaning humidifier 1 according to the embodiment, the disc assembly 30 is arranged to surround the ventilation fan 42 in a manner that a center of rotation A of the disc assembly 30 is almost aligned with a center of rotation B of the ventilation fan 42.

The disc assembly 30 may include a first side plate 31 adjoining the rear cover 13 when received in the main body 11, a second side plate 32 adjoining the front cover 12 when received in the body frame 11, and a plurality of discs 33 disposed between the first and the second side plates 31 and 32.

An opening 30a is formed through the first side plate 31, the second side plate 32, and the plurality of discs 33 to have a greater diameter than an outer diameter of the ventilation fan 42. In other words, the disc assembly 30 substantially has a ring shape and is connected with the main body 11 as the ventilation fan 42 is inserted in the opening 30a of the disc assembly 30. More specifically, the disc assembly 30 is received in the main body 11 so that a thickness portion T1 of the disc assembly 30 is overlapped with a thickness portion T2 of the ventilation fan 42. The discs 33 include a plurality of disc members deposited at predetermined intervals. The discs 33 may each have a penetration hole (not shown) corresponding to a connection member 31a formed at the first side plate 31 such that the connection member 31a is fitted around the connection member 31a. The second side plate 32 may also have a penetration hole (not shown) corresponding to the connection member 31a to be fit around the connection member 31a. In addition, spacers 34 are provided among the first side plate 31, each of the discs 33, and the second side plate 32, thereby adjusting the interval between the respective discs 33.

The disc assembly 30 is connected to a driving unit 70 that generates a rotational force at the disc assembly 30. To this end, the driving unit 70 may include a driving motor 71, and a driving gear 72 connected with a gear unit 32a of the second side plate 32. As the driving gear 72 of the driving unit 70 is engaged with the gear unit 32a of the second side plate 32, the driving force of the driving unit 70 is transmitted to the disc assembly 30.

The driving gear 72 may be disposed lower than the center of rotation A of the disc assembly 30 in order to avoid interference with the driving unit 70 during connection and separation of the disc assembly 30 with respect to the air cleaning humidifier 30.

Additionally, a supporting member 80 may be formed inside the water tub 20 to support the disc assembly 30. The supporting member 80 may be removably provided at both inner sides of the water tub 20. The supporting member 80 is rotatably formed to support the disc assembly 30 and guide the disc assembly 30 to be more stably rotated by the driving gear 72. The support member 80 may include a roller disposed higher than a water level.

Furthermore, a cut portion 30b is formed at the disc assembly 30 so as to prevent deterioration of the velocity of the air current generated by the ventilation fan 42. The cut portion 30b may be formed by cutting out a part of circumferences of the first side plate 31, to form a space S, the second side plate 32 and the discs 33 substantially by a fan-shape.

More specifically, the cut portion 30b is formed by cutting out a part of a ring portion 30c constituting the circumferential body of the disc assembly 30. Therefore, the cut portion 30b includes a first sectional surface P1 and a second sectional surface P2 formed into a rectangular plane by partially cutting out the ring portion 30c of the disc assembly 30. A cut-out area of the disc assembly 30 is thus generated between the first and the second sectional surfaces P1 and P2.

By passing through the cut portion 30b of the disc assembly 30, the air current generated by the ventilation fan 42 may be moved to the air discharge port 14c with a reduced resistance.

The cut portion 30b of the embodiment has a fan-shape according to the drawings. However, considering that the cut portion 30b formed by partially cutting the ring portion 30c aims to prevent deterioration of the velocity of the air current moving to the air discharge port 14c, it will be readily understood by those skilled in the art that the cut portion 30b may have any other shape designed through analysis of the air current.

The driving unit 70 rotates the disc assembly 30 in a manner that the cut portion 30b of the disc assembly 30 is not submerged in the water tub 20 because the humidifying performance may be deteriorated if the cut portion 30b which is incapable of absorbing water is submerged in the water tank 20. The rotational operation of the disc assembly 30 will be explained later.

Hereinafter, the operation of the air cleaning humidifier according to the embodiment will be described.

First, when the air cleaning humidifier 1 is started, water stored in the water tank 50 is supplied to the water tub 20. The water supplied from the water tank 50 is stored in the water receiving part 21 passing through the water supply part 22. Here, quantity of the water stored in the water receiving part 21 may be adjusted to have a regular water level 21a. Part of the disc assembly 30 is submerged in the water because the ring-shaped disc assembly 30 is disposed to be partly lower than the water level 21a of the water tub 21.

When the user selects an air cleaning and humidifying mode through the operation unit 12a, the ventilation fan 42 and the disc assembly 30 are simultaneously driven. Therefore, cleaned air is passed through the disc assembly 30 which is rotating and is then discharged out through the air discharge port 14c.

The process of the air cleaning and humidifying mode will be more specifically described. The driving force of the driving unit 70 is transmitted from the driving gear 72 to the disc assembly 30, and accordingly the disc assembly 30 is rotated.

Since the ventilation fan 42 is inserted in the opening 30a of the disc assembly 30 having the ring shape, the disc assembly 30 is rotated along the outer circumference of the ventilation fan 42. The rotation of the ventilation fan 42 generates an air current within the air cleaning humidifier 1. Specifically, the air current is formed as air is drawn in through the air suction port 13a formed on the rear surface of the air cleaning humidifier 1, passed through the filter 60, drawn into the ventilation fan 42 and then discharged to the outside through the disc assembly 30.

That is, since the thickness portion of the ventilation fan 42 and the thickness portion of the discs 33 are overlapped with each other, the air current discharged from the ventilation fan 42 is blown to the disc assembly 30. While the blown air current is passing through the plurality of discs 33, the water soaked in the discs 33 is vaporized, thereby humidifying the air current. The humidified air is discharged out of the body frame 10 through the air discharge port 14c.

Here, since the disc assembly 30 has the cut portion 30b formed by cutting a part of the ring portion 30c, the air current generated by the ventilation fan 42 may cause less interference with or resistance to the disc assembly 30 when moving to the air discharge port 14c.

The disc assembly 30 is driven in a state wherein only the ring portion 30c of the disc assembly 30 is submerged in the water tub 20 but the cut portion 30b is not submerged.

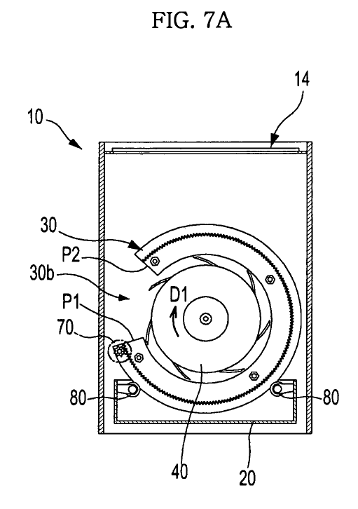

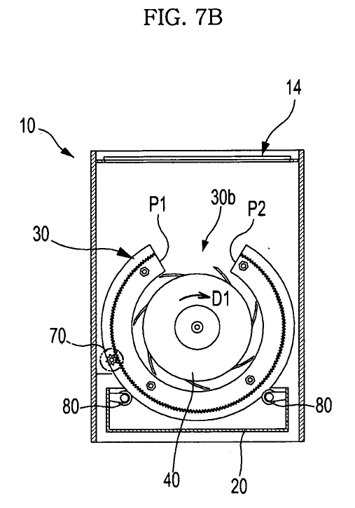

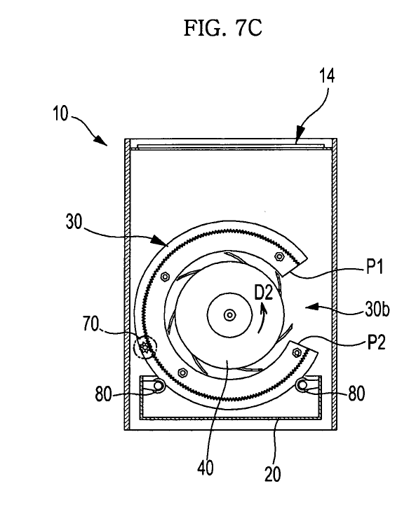

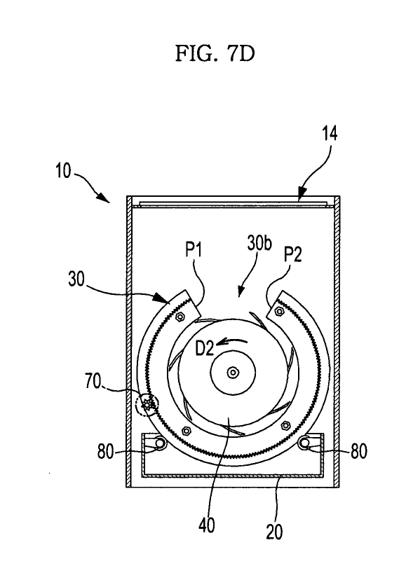

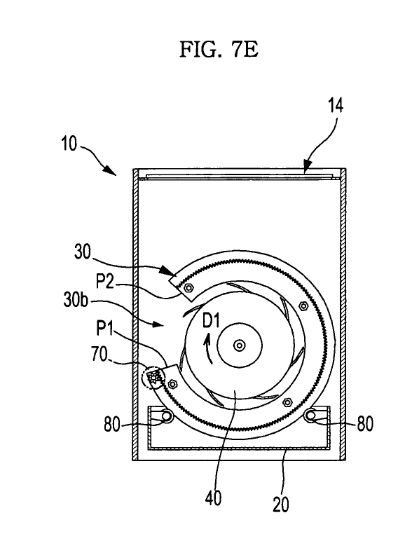

That is, when the driving unit 70 approaches the first sectional surface P1 of the cut portion 30b as shown in FIG. 7A, the driving unit 70 rotates the disc assembly 30 by about 120° in a forward direction D1 so that the cut portion 30b of the disc assembly 30 approaches nearest to the discharge grill 14 as shown in FIG. 7B.

From the state of FIG. 7B, the driving unit 70 further rotates the disc assembly 30 by about another 120° in the forward direction D1 so that the second sectional surface P2 of the cut portion 30b approaches the water level 21 a in the water tub 20 as shown in FIG. 7C.

When the disc assembly 30 is rotated to the position as shown in FIG. 7C, the driving unit 70 transmits the driving force to rotate the disc assembly 30 in a backward direction D2, so that the cut portion 30b of the disc assembly 30 approaches nearest to the discharge grill 14 as shown in FIG. 7D.

When the disc assembly 30 is disposed as shown in FIG. 7D, the driving unit 70 rotates the disc assembly 30 again by about 120° in the backward direction D2 so that the first sectional surface P1 of the cut portion 30b approaches the water level 21a in the water tub 20 as shown in FIG. 7E.

Accordingly, when the first sectional surface P1 approaches the driving unit 70 as shown in FIG. 7E, the driving unit 70 rotates the disc assembly 30 again by about 120° in the forward direction D1 and repeatedly performs the above-described processes.

The driving unit 70 may include a step motor in order to rotate the disc assembly 30 in the forward direction D1 and then in the backward direction D2 in accordance with input angles.

According to the above structure, the cut portion 30b of the disc assembly 30 is always maintained out of the water tub 20 during rotation of the disc assembly 30 in the forward direction D1 or backward direction D2. Therefore, deterioration of the humidifying performance of the disc assembly 30 caused when the cut portion 30b is submerged in the water tub 20 may be prevented.

If the air cleaning mode for only air cleaning is selected, a process of correcting the position of the disc assembly 30 may be further performed so that the cut portion 30b is disposed nearest to the air discharge port 14c. This is performed to minimize interference between the air current generated by the ventilation fan 42 and the disc assembly 30. Accordingly, volume of the air current is increased and the air current is quickly supplied to the interior space.

As is apparent from the above description, an air cleaning humidifier according to the embodiment is capable of minimizing interference between an air current generated at a ventilation fan and a disc assembly by forming a cut portion at the disc assembly.

Although a few embodiments have been shown and described, it would be appreciated by those skilled in the art that changes may be made in these embodiments without departing from the principles and spirit of the invention, the scope of which is defined in the claims and their equivalents.

What is claimed is:

1. An air cleaning humidifier comprising:
a body frame having an air discharge port;
a water tub disposed in the body frame;
a ventilation fan generating an air current in the body frame; and
a disc assembly arranged around the ventilation fan and rotated with a part thereof submerged in the water tub,
wherein the disc assembly includes a ring portion forming a circumferential body thereof and a cut portion formed by cutting out a part of the ring portion and disposed adjacent the air discharge port.

2. The air cleaning humidifier according to claim 1, wherein the cut portion is formed by cutting out a part of the ring portion in a fan-shape.

3. The air cleaning humidifier according to claim 1, further comprising a driving unit driving the disc assembly to transmit a rotational force to the disc assembly.

4. The air cleaning humidifier according to claim 3, wherein the disc assembly includes a side plate comprising a gear unit, and
the driving unit is engaged with the gear unit.

5. The air cleaning humidifier according to claim 1, wherein the driving unit rotates the disc assembly forward and backward so that the ring portion of the disc assembly is submerged in the water tub while the cut portion is not submerged.

6. The air cleaning humidifier according to claim 1, further comprising a supporting member formed in the water tub to support the disc assembly.

7. The air cleaning humidifier according to claim 1, wherein a center of rotation of the disc assembly is axially aligned with a center of rotation of the ventilation fan, and
the disc assembly is received in the body frame so that thickness portions of the disc assembly and the ventilation fan are overlapped with each other.

8. The air cleaning humidifier according to claim 1, wherein the disc assembly includes an opening having a greater diameter than an outer diameter of the ventilation fan so that the disc assembly fan may be inserted in the disc assembly.

9. The air cleaning humidifier according to claim 1, wherein the disc assembly includes at least two side plates, a plurality of discs disposed between the side plates, and spacers to maintain predetermined intervals among the discs.

10. The air cleaning humidifier according to claim 1, wherein the body frame includes an air suction port formed at a rear surface thereof to draw air.

11. The air cleaning humidifier according to claim 1, wherein the cut portion is at a position nearest to the air discharge port when an air cleaning mode is selected.

12. The air cleaning humidifier according to claim 1, wherein the body frame further includes a heater to heat the air humidified by the disc assembly.

13. The air cleaning humidifier according to claim 1, wherein the body frame further includes a sterilizer to sterilize the air humidified by the disc assembly.

14. An air cleaning humidifier comprising:
a body frame having an air suction port and an air discharge port;
a water tub connected to a lower part of the body frame;
a disc assembly rotated while partially submerged in the water tub; and
a ventilation fan rotated in the state of being aligned with a center of rotation of the disc assembly, to draw in air through the air suction port and discharge the air through the air discharge port,
wherein the disc assembly includes a cut portion formed by cutting out a part of a circumference of the disc assembly so that an air current generated by the ventilation fan is smoothly moved to the air discharge port.

15. The air cleaning humidifier according to claim 14, further comprising a driving unit that rotates the disc assembly forward and backward so that the cut portion of the disc assembly is not submerged in the water tub.

16. The air cleaning humidifier according to claim 15, wherein the driving unit includes a step motor which rotates the disc assembly forward and then backward by a predetermined input angle.
